(19) United States
(12) Plant Patent Application Publication
(10) Pub. No.: US 2019/0133016 P1
(43) Pub. Date: May 2, 2019

(54) Begonia plant named 'Citra'

(50) ;

(71) Applicant: Terra Nova Nurseries, Inc., Canby, OR (US)

(72) Inventor: John C. Pavlich, Canby, OR (US)

(73) Assignee: Terra Nova Nurseries, Inc., Canby, OR (US)

(21) Appl. No.: 15/732,367

(22) Filed: Oct. 31, 2017

(51) Int. Cl. A01H 6/18

(52) U.S. Cl. PLT/343

(57) ABSTRACT

A new and distinct form of Begonia plant characterized by numerous, large, lemon-gold flowers edged in Chinese red, continuous bloom time, semi-double to double flowers, pine green leaves, a trailing habit, and excellent vigor.

Botanical denomination: Begonia hybrid.

Variety designation: ‘Citra’.

### BACKGROUND OF THE INVENTION

The present invention relates to a new and distinct cultivar of Begonia, of the family Begoniaceae, and given the cultivar name, ‘Citra’. Begonia ‘Citra’ is a hybrid involving breeding lines using Begonia tuberhybrida, Begonia boliviensis and unnamed rhizomatous Begonia. The new cultivar originated from a controlled cross of unnamed and unreleased proprietary seedlings.

This selection is uniquely characterized by:
1. numerous, large, lemon-gold flowers edged in Chinese red,
2. continuous bloom time,
3. semi-double to double flowers,
4. pine green leaves,
5. a trailing habit, and
6. excellent vigor.

Compared to Begonia ‘Yachbon’ (Bon Bon Cherry), an unpatented plant, the new cultivar has lemon-gold flowers edged in Chinese red and pine green leaves rather than cherry colored flowers with green leaves.

The new variety has been reproduced only by asexual propagation (leaf and stem cuttings and tissue culture using growing tips). Each of the progeny exhibits identical characteristics to the original plant. Asexual propagation by micropropagation as done in Canby, Oreg., shows that the foregoing characteristics and distinctions come true to form and are established and transmitted through succeeding propagations. The present invention has not been evaluated under all possible environmental conditions. The phenotype may vary with variations in environment without a change in the genotype of the plant.

### BRIEF DESCRIPTION OF THE DRAWINGS

FIG. 1 shows a 12 month old plant of the new cultivar in 10 inch pot in a warm greenhouse in April in full bloom in Canby, Oreg.

FIG. 2 shows a close up of the same plant shown in FIG. 1.

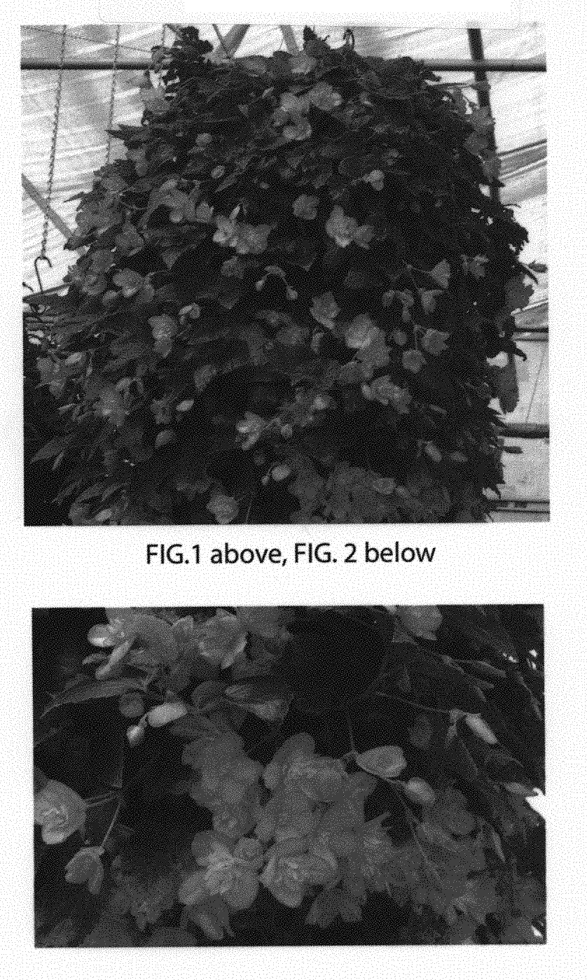

### DETAILED PLANT DESCRIPTION

The following is a detailed description of the new Begonia cultivar based on observations of a 7-month-old specimen grown in a 10 inch hanging basket container in a warm greenhouse in September in Canby, Oreg. The color descriptions are all based on The Royal Horticultural Society Colour Chart, 5^{th} edition, 2007.
Plant:
   Type.—Herbaceous, rhizomatous perennial.
   Form.—Decumbent.
   Cold hardiness.—USDA Zone 9-11.
   Size.—Grows to 49 cm wide and 68 cm deep.
   Vigor.—Excellent.
   Roots.—Rooting initiates in two weeks, roots Orange 27B in color.
Stem:
   Type.—Decumbent.
   Size.—Grows to 41 cm long and 7 mm wide.
   Internodes.—From 5 mm to 26 mm long.
   Surface.—Pubescent.
   Color.—Yellow-Green 148C fading to Greyed-Red 181D at the distal end of stems.
Leaf:
   Type.—Simple.
   Arrangement.—Alternate.
   Shape.—Obliquely lanceolate, asymmetric.
   Leaf strength.—Weak.
   Blade size.—Grows to 5 cm wide and 11 cm long.
   Margins.—Biserrate.
   Apex.—Acuminate.
   Base.—Oblique.
   Venation.—Palmate, veins Green 139D topside, bottom side Green 139B.
   Surface texture.—Pubescent on top; bottom side is glabrous, moderately pubescent along veins.
   Stipules.—Deciduous, ovate, 4 mm long and 6 mm wide, apex acute, base clasping, erose, both sides glabrous and Yellow-Green 145B.
   Petiole description.—Grows to 3 cm long and 2 mm wide, succulent, pubescent, Greyed-Red 181D.
   Leaf color.—New leaves top side Green 137C, bottom side Greyed Purple 183B; old leaves top side Green 139A, bottom side Green 138A tinged with Greyed-Red 180B.
Inflorescence:
   Type.—Axillary, bisexual, cymose.
   Number of flowers per cyme.—3.
   Peduncle description.—Grows 5 cm long and 4 mm wide, glabrous, weak, Greyed-Orange N170B; with 2 opposite, dehiscent petiolate leaf-bracts below each cyme, broadly ovate, grow to 8 mm wide and 5 mm long, margins ciliate, tip obtuse to notched, base clasping, both sides glabrous, both sides Yellow Green 145A lightly tinted Greyed Purple 187A on top margin.
   Pedicel description.—Grows to 36 mm long, 2 mm wide, glabrous, medium strength, Greyed Red 179B on top side, bottom Greyed-Orange 170C.
   Bloom period.—Year round in Canby, Oreg.
Flower bud:
   Size.—Staminate flower bud to 28 mm long and 25 mm wide, pistillate flower bud to 33 mm long and 20 mm wide.
   Shape.—Flat oval.
   Surface texture.—Glabrous.
   Color.—Orange-Red 34A on top half blending to Yellow-Green 145B on bottom, Yellow Green N144C on ovary.
Flower:
   Type.—Monoecious, bilaterally symmetrical.
   Pistillate flower.—Grows to 20 mm deep and 46 mm wide overall; 5 fleshy tepals, each grow to 24 mm long and 21 mm wide, ovate, tip obtuse to acute, base between cordate and truncate, margins entire, glabrous on both sides, top side Yellow 6D at base blending into a margin encompassing 1 mm to 5 mm of Orange-Red 32A, bottom side Orange-Red 31D blending to Orange-Red 32A; 1 pistil, to 20 mm long overall, ovary 9 mm long and 15 mm wide, three winged (two short and one longer), Yellow-Green N144C, style 2-branched, 6 mm long and 4 mm wide overall, stigma 2-branched and twisted, each stigma 4 mm deep and 1.5 mm wide, Yellow-Orange 23B.
   Staminate flower.—Semi-double to double, grows to 25 mm deep and 53 mm wide overall; with 4 main tepals, 2 outer tepals grow to 31 mm long and 29 mm wide, obovate, margin entire, base truncate to cordate, tip retuse to obtuse, top side Yellow 3C at base blending to a margin encompassing 1 mm to 5 mm of Orange-Red 32A, bottom side Orange-Red 32A, glabrous on both sides; 2 inner tepals, grow to 34 mm long and 19 mm wide, both sides glabrous, obelliptic, margin entire, base attenuate, tip acute to obtuse, top side Yellow 3C from center blending to a margin encompassing 1 mm to 3 mm of Orange-Red 32A, bottom side Orange-Red 33A blending to Orange-Red 33C near the base; 10-24 central tepals modified from stamens grow to 24 mm long and 9 mm wide, both sides glabrous, broadly oblanceolate, margin entire, base obovate, tip obtuse, top side Yellow 9B from base blending to a margin encompassing 1 mm to 3 mm of Orange-Red 32A; no stamen, no fertile pollen, male sterile.
   Fragrance.—None.
   Lastingness.—A cyme blooms for about 2 weeks on the plant, flowers are self-cleaning.
Fruit and seed: none seen.
Diseases and pests: The new cultivar is typical to the genus. No known resistances to pests or diseases. No problems have been found in Canby, Oreg.

I claim:

1. A new and distinct form of Begonia plant as hereby illustrated and described.
